(19) United States
(12) Patent Application Publication
(10) Pub. No.: US 2010/0106234 A1
(43) Pub. Date: Apr. 29, 2010

(54) Medical Devices With Extended Drug Diffusion Pathway

(75) Inventor: Robert J. Melder, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/256,668

(22) Filed: Oct. 23, 2008

(51) Int. Cl. A61F 2/86

(52) U.S. Cl. 623/1.11

(57) ABSTRACT

A system for treating a vascular condition includes a catheter, a stent disposed on the catheter, the stent having a stent framework including a stent wire and at least one stent foil attached to and wrapped around the stent wire, and a therapeutic agent coating disposed on an inner surface of the at least one stent foil. A method of manufacturing a stent includes securing a first edge of a stent foil to a stent wire, applying a therapeutic agent coating to an inner surface of the stent foil and wrapping the coated stent foil around the stent wire to encase the coating between the stent foil and the outer surface of the stent wire, securing a second edge of the wrapped stent foil to the stent wire, forming at least one exit port along the secured second edge and forming the wrapped stent wire into a stent framework.

### TECHNICAL FIELD

This invention relates generally to biomedical devices. More specifically, the invention relates to implantable stents and other medical devices having prolonged delivery of therapeutic agents.

### BACKGROUND OF THE INVENTION

Implantable devices often include a therapeutic agent as part of a therapeutic agent coating or as part of the material forming the implantable device. With these devices, the therapeutic agent contained in the coating begins to elute or diffuse from the device surface soon after implantation resulting in a burst of therapeutic agent at the treatment site. In some situations an initial burst of therapeutic agent may be useful. However, in other situations a sustained, controlled release of the therapeutic agent from the device surface is desired.

Polymers, mixed with the therapeutic agent, have been used to control the release rate of the therapeutic agent from the device surface. Where a polymer is used, an initial burst of therapeutic agent occurs immediately after implantation followed by a slow sustained release over a predetermined length of time. The length of time that a therapeutic agent elutes is often critical to successful treatment of the patient's condition. In many cases a slow release over an extended period of time is desirable. However, the length of time that the therapeutic agent is eluted often depends on factors such as the presence of a polymer in the coating, the type of polymer(s) used and the thickness of the drug or drug/polymer coating and the amount of drug in the coating.

In the case of diffusion from a therapeutic coating including a polymer, delivery of drug to a surrounding matrix is regulated by the effective diffusion coefficient of the drug in the polymer and the length of the mean diffusion path that a molecule must take to exit the polymer. The mean diffusion path may be considered to be half the thickness of the coating. In the case of drug dissolution into solution from a coating without a polymer, the diffusion path may be extremely short since rapid hydration of an applied drug formulation may quickly render the entire drug load soluble and subject to convective transport into the surrounding matrix.

In an effort to increase the mean diffusion path of the drug or drugs contained within the coating, thicker therapeutic agent coatings have been applied to medical device surfaces, thereby extending the duration of elution. Factors to consider when applying a thicker coat include the device to be coated, the size and/or crossing profile of the device to be coated and the pathway the device must traverse to reach the treatment site. A thicker coating may be applied as one layer or in multiple layers as best suited for the particular application. However, thicker coatings have an increased chance of damage during every stage of the implantation procedure. Therefore, the effectiveness of the increase of the mean diffusion path of the drug by increasing the coating thickness is limited.

Some coating procedures provide barrier layers between drug or drug-polymer layers to separate layers having different drugs. Barrier layers also may be used to increase the elution time and to effectively increase the mean diffusion path by slowing down the degradation of the coating. One drawback to this is that the barrier layers may not be suitable due to an unwanted increase in thickness of the coating and, thus, the crossing profile. Another drawback is that the barrier layer may not keep the different drugs from comingling prior to elution from the device.

It would therefore be desirable, to provide an implantable therapeutic agent eluting medical device that would overcome the limitations and disadvantages described above.

### SUMMARY OF THE INVENTION

One aspect of the present invention provides a system for treating a vascular condition. The system includes a catheter, a stent disposed on the catheter, the stent having a stent framework including a stent wire and at least one stent foil attached to and wrapped around the stent wire, and a therapeutic agent coating disposed on an inner surface of the at least one stent foil.

Another aspect of the invention provides a stent for treating a vascular condition. The stent includes a stent framework including a stent wire and at least one stent foil attached to and wrapped around the stent wire and a therapeutic agent coating disposed on an inner surface of the at least one stent foil.

Another aspect of the invention provides a method of manufacturing a stent for treating a vascular condition. The method includes securing a first edge of at least one stent foil to a stent wire, applying a therapeutic agent coating to an inner surface of the at least one stent foil and wrapping the therapeutic agent coated stent foil around an outer surface of the stent wire to encase the therapeutic agent coating between the inner surface of the stent foil and the outer surface of the stent wire. The method further includes securing a second edge of the wrapped stent foil to the stent wire, forming at least one exit port along the secured second edge and forming the wrapped stent wire into a stent framework.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of a system for treating a vascular condition comprising a therapeutic agent carrying stent coupled to a catheter, in accordance with one embodiment of the present invention;

FIGS. 2A to 2D illustrate one embodiment of a stent framework of a stent suitable for use in the system illustrated in FIG. 1, in accordance with the present invention;

FIGS. 3A to 3C illustrate serial cross sections of another embodiment of a stent framework of a stent suitable for use in the system illustrated in FIG. 1, in accordance with the present invention;

FIG. 4 illustrates a cross section of another embodiment of a stent framework of a stent suitable for use in the system illustrated in FIG. 1, in accordance with the present invention;

FIG. 5 illustrates a cross section of another embodiment of a stent framework of a stent suitable for use in the system illustrated in FIG. 1, in accordance with the present invention; and

FIG. 6 is a flow diagram of a method for manufacturing a stent for treating a vascular condition, in accordance with the present invention.

### DETAILED DESCRIPTION

The invention will now be described by reference to the figures wherein like numbers refer to like structures. The terms “distal” and “proximal” are used herein with reference to the treating clinician during the use of the catheter system; “distal” indicates an apparatus portion distant from, or a direction away from the clinician and “proximal” indicates an apparatus portion near to, or a direction towards the clinician.

The present invention is directed to a system for treating abnormalities of the cardiovascular system comprising a catheter and a therapeutic agent-carrying stent disposed on the catheter. Those with ordinary skill in the art will appreciate that the below described invention can be applied to other implantable medical device composed of wire, such as, for example, pacemaker leads.

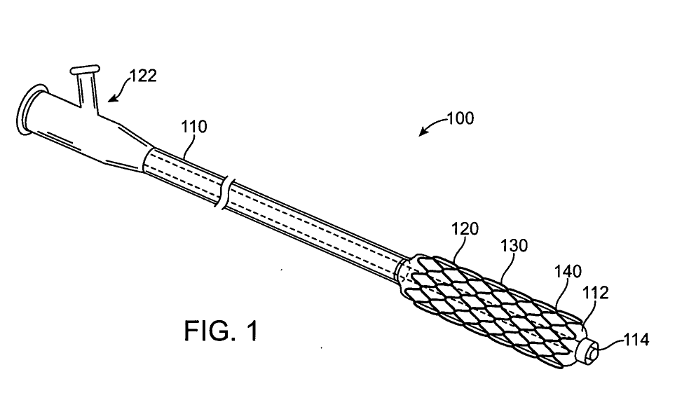

FIG. 1 illustrates on embodiment of a system 100 for treating a vascular condition. System 100 comprises therapeutic agent carrying stent 120 coupled to catheter 110. Catheter 110 includes a balloon 112 that expands and deploys stent 120 within a vessel of the body. After positioning 120 within the vessel, balloon 112 is inflated by pressurizing a fluid such as a contrast fluid or saline solution that fills a lumen inside catheter 110 and balloon 112. Stent 120 is expanded until a desired diameter is reached; then the contrast fluid is depressurized or pumped out, separating balloon 112 from stent 120 and leaving stent 120 deployed in the vessel of the body. Alternately, catheter 110 may include a sheath that retracts to allow expansion of a self-expanding embodiment of stent 120.

Catheter 110 may comprise an elongated tubular member having a substantially circular cross-section and inside and outside walls that are substantially smooth. Catheter 110 may be secured at its proximal end to a suitable Luer fitting 122 and may include a distal rounded end to reduce harmful contact with a vessel. Catheter 110 may be manufactured substantially from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax®, Vestamid®, Tecoflex®, Halar®, Hyflon®, Pellathane®, combinations thereof, and the like. Catheter 110 may include lumen 114 formed therethrough allowing it to be advanced over a pre-positioned guidewire.

Balloon 112, shown in a collapsed state, may be any variety of balloon capable of expanding stent 120. Balloon 112 may be manufactured from any sufficiently elastic material such as polyethylene, polyethylene terephthalate (PET), nylon, or the like.

Stent 120 includes stent framework 130 forming interior and exterior surfaces of the stent. Stent framework 130 includes an elongated stent wire and at least one foil wrapped around an outer surface of the wire.

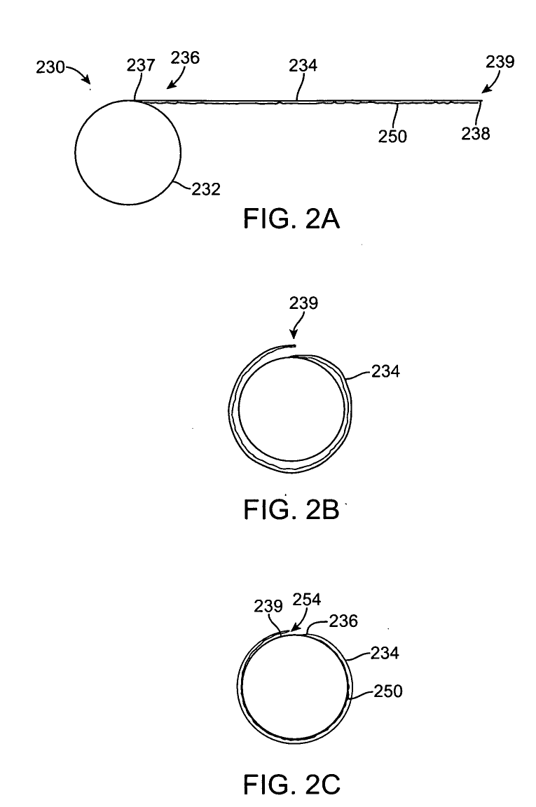

FIGS. 2A to 2C illustrate a cross section of one embodiment of stent framework 230, in accordance with the present invention. FIG. 2D illustrates a perspective view of a portion of stent framework 230. Stent framework 230 may be used for forming a drug eluting medical device such as stent 120. Stent framework 230 comprises stent wire 232 and stent foil 234. Stent wire 232 comprises a biocompatible metal or metal alloy. In one embodiment of the invention, the stent wire 232 comprises one or more of a variety of biocompatible metals such as stainless steel, titanium, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys. The metal composition of stent wire 232 gives the stent framework the mechanical strength to support the lumen wall of the vessel and sufficient longitudinal flexibility so that it can be transported through the cardiovascular system.

Stent foil 234 comprises a biocompatible metal or metal alloy. In one embodiment of the invention, the stent foil 234 comprises one or more of a variety of biocompatible metals such as stainless steel, titanium, magnesium, aluminum, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys. In one embodiment, stent wire 232 and stent foil 234 are composed of the same material. In one embodiment, stent wire 232 and stent foil 234 are composed of nitinol. In another embodiment, stent wire 232 and stent foil 234 are composed of stainless steel.

Stent framework 230 is formed by first attaching a first edge 236 of stent foil 234 along the length of stent wire 232. In one embodiment, first edge 236 is attached to stent wire 232 by welding a continuous weld 237 along the length of stent wire 232. In another embodiment, first edge is attached to stent wire using an adhesive. Those with skill in the art will appreciate that stent foil 234 may be attached to stent wire 232 by any means suitable for providing a continuous attachment.

Stent framework also includes at least one therapeutic agent coating 250. Therapeutic agent coating 250 comprises a biologically or pharmacologically active substance. In one embodiment, the biologically or pharmacologically active substance may be suspended in a polymer matrix or carrier. In one embodiment, the polymer matrix or carrier is biodegradable or bioresorbable such that it is absorbed in the body. The polymer matrix may comprise biodegradable polymers such as polylactic acid (PLA), polyglycolic acid, and their copolymers, polyethylene oxide (PEO), caproic acid, polyethylene glycol, polyanhydrides, polyacetates, polycaprolactones, poly(orthoesters), polyamides, polyurethanes and other suitable polymers

The term “biologically or pharmacologically active substance” refers to any substance, whether synthetic or natural, that has a pharmacological, chemical, or biological effect on the body or a portion thereof. Suitable biologically or pharmacologically active materials that can be used in embodiments of the present invention include without limitation glucocorticoids (e.g. dexamethasone, betamethasone), antithrombotic agents such as heparin, cell growth inhibitors, hirudin, angiopeptin, aspirin, growth factors such as VEGF, antisense agents, anti-cancer agents, fibrinolytics, antirestenotic agents, and anti-inflammatory agents may be used. Antiplatelet agents can include drugs such as aspirin and dipyridamole. Aspirin is classified as an analgesic, antipyretic, anti-inflammatory and antiplatelet drug. Dipyridamole is a drug similar to aspirin in that it has anti-platelet characteristics. Dipyridamole is also classified as a coronary vasodilator. Anticoagulant agents may include drugs such as heparin, protamine, hirudin and tick anticoagulant protein. Anti-cancer agents may include drugs such as taxol and its analogs or derivatives. Taxol is also classified as a cell-growth inhibitor. Antioxidant agents may include probucol. Antiproliferative agents may include drugs such as amlodipine, doxazosin, and sirolimus or other—limus family compounds. Antimitotic agents and antimetabolite agents may include drugs such as methotrexate, azathioprine, vincristine, vinblastine, 5-fluorouracil, adriamycin and mutamycin. Antibiotic agents can include penicillin, cefoxitin, oxacillin, tobramycin, and gentamicin. Suitable antioxidants include probucol. Also, genes or nucleic acids, or portions thereof may be used. Recombinant DNA products, or other bioactive agents, diagnostic agents, radioactive isotopes, or radiopaque substances may be used depending on the anticipated needs of the targeted patient population. Such genes or nucleic acids can first be packaged in liposomes or nanoparticles. Furthermore, collagen-synthesis inhibitors, such as tranilast, may be used.

The therapeutic agent coating 250 containing the at least one therapeutic agent may additionally contain excipients including solvents or other solubilizers, stabilizers, suspending agents, antioxidants, and preservatives, as needed to deliver an effective dose of the therapeutic agent to the treatment site. The therapeutic agent coating 250 may be applied to the outer surface of stent wire 232 and/or an inner surface 238 of stent foil 234 by any means known in the art such as, for example, by spraying, dipping, and brushing. In one embodiment, the coating is applied as a liquid by brushing or spraying, and then dried to remove solvent using air, vacuum, or heat, and any other effective means of causing the formulation to adhere to the stent framework. In one embodiment, the therapeutic agent coating is applied to the stent foil prior to the attachment of the stent foil to the stent wire.

In one embodiment illustrated in FIG. 2A, a liquid therapeutic agent formulation is sprayed on the inner surface 238 of stent foil 234, and forms a therapeutic agent coating 250 having a uniform thickness. If needed, therapeutic agent coating 250 is cured by exposure to ultraviolet light, heat, gamma irradiation or any other appropriate means.

Referring to FIG. 2B, after application of therapeutic agent coating 250 to stent foil 234, stent foil 234 is wrapped around at least a portion of the outer surface of stent wire 232. In this embodiment, the width of stent foil 234 approximates the circumference of stent wire 232. As will be discussed below, the width of stent foil may vary depending on a particular application or a particular predetermined diffusion pathway.

Stent foil 234 is wrapped around stent wire 232 in such a manner as to bring second edge 239 in close proximity to attached first edge 236. As shown in FIG. 2C, second edge 239 is attached adjacent but not touching or over lapping first edge 236. Second edge 239 is secured to stent wire 232. Wrapping stent foil 234 around stent wire 232 and securing second edge 239 to stent wire 232 effectively encases the therapeutic agent within stent framework 230 formed by stent foil 234 and stent wire 232. Second edge 239 may be secured by a tack weld applied at intervals along the length of stent wire 232. In another embodiment, stent foil 234 including second edge 239 is secured to stent wire 232 by the adhesive properties of the therapeutic agent coating 250. In another embodiment, second edge 239 is attached to stent wire 232 by adhesive disposed at intervals along the length of stent wire 232. Securing second edge 239 at intervals provides a plurality of therapeutic agent exit ports 254 along the length of stent wire 232. After implantation, the at least one therapeutic agent contained within coating 250 exits stent 120 through exit ports 254.

The foil wrapped wire having an encased therapeutic agent can be formed into a drug eluting medical device such as, for example, stent 120. In one embodiment, stent framework 230 is formed into stent 120 by shaping the foil wrapped metallic wire. The foil wrapped wire may be shaped into a stent by any means known in the art.

In this embodiment, the mean diffusion pathway of the encased therapeutic agent is extended well beyond a mean diffusion pathway provided by a coating disposed directly on a stent surface and exposed to the vessel wall. In an example, a stent wire 0.0050 inches in diameter with a coating of 10 μm in thickness and a foil approximating the circumference of the stent wire, at least a 40-fold increase in the mean diffusion path is provided.

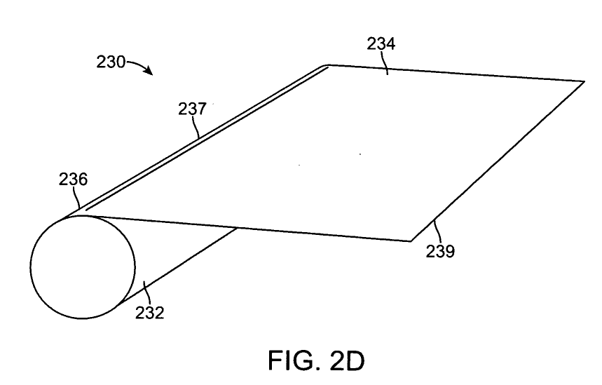

FIG. 3A to 3C illustrate serial cross sections of another embodiment of a stent framework 330 made in accordance with the present invention. In treating various conditions it is often required to administer more than one drug or therapeutic agent. Due to certain properties of various drugs and other types of therapeutic agents it is desirable to segregate one drug from another until such time as the drug is administered. Stent framework 330 provides a structure for separating one drug from another in a single drug delivery device.

Aspects of stent framework 330 similar to stent framework 130 and 230 will not be discussed further. In this embodiment, stent framework includes three stent foils 334A, 334B and 334C wrapped around stent wire 332 as shown in FIG. 3C. As shown in FIG. 3A, first edge 336 of each stent foil 334A to 334C is attached to stent wire 332. First edges 336 may be attached by weld, adhesive or any other means described above. After attachment of first edges 336 to stent wire 332, a therapeutic agent coating 350A is applied to stent foil 334A. Stent foil 334A is wrapped around stent wire 332 as shown in FIG. 3B. A second edge 339A may be attached to stent wire 332 as described above. Each second edge of stent foils 334A to 334C may be attached to stent wire 332 upon completion of the wrapping or, alternatively, a tack weld may be applied after all stent foils 334A to 334C are wrapped around stent wire 332.

Referring to FIG. 3B, after stent foil 334A is wrapped around stent wire 332 a second therapeutic coating 350B is applied to stent foil 334B. Wrapping of stent foil 334B encases the second therapeutic agent coating between the inner surface of second stent foil 334B and the outer surface of stent foil 334A. Wrapping of stent foil 334B exposes the inner surface of stent foil 334C at which time a third therapeutic agent coating 350C may be applied. Wrapping of stent foil 334C encases the third therapeutic agent coating between the inner surface of third stent foil 334C and the outer surface of stent foil 334B. Those with skill in the art will appreciate that the number of stent foils may vary depending on the particular application. Factors to consider are the number of drugs or agent to be administered and the dimensions of the vascular site to be accessed and treated. It will be appreciated that the embodiment illustrated in FIGS. 3A to 3C may be used to isolate one therapeutic agent from another as may be required by the characteristics of the therapeutic agents to be administered. It will also be appreciated that more than one layer can contain the same therapeutic agent or drug in an effort to increase the amount of therapeutic dose. In one embodiment, the therapeutic agent contained within therapeutic agent coatings 350A to 350C is different. In another embodiment, the therapeutic agent contained within therapeutic agent coatings 350A to 350C is the same.

As shown in FIG. 3C, the exit ports for the therapeutic agents within therapeutic agent coatings 350A to 350C are aligned to provide a single locus from which the drugs exit. This alignment provides a similar mean diffusion path for the therapeutic agents contained therein. Based on the above disclosure, one with skill in the art will appreciate that the mean diffusion path may be adjusted by changing the width of the stent foil wrapped around the stent wire, thus changing the location of the exit port associated with that particular stent foil. Illustrated are exit ports having a single location that would deliver drugs into the vessel wall or into the lumen. In other embodiments, the width of each stent foil 334A to 334C may be chosen to provide directional delivery of the drugs. In other embodiment, the width of the stent foils is adjusted to provide an exit port at other locations around the circumference of the stent wire.

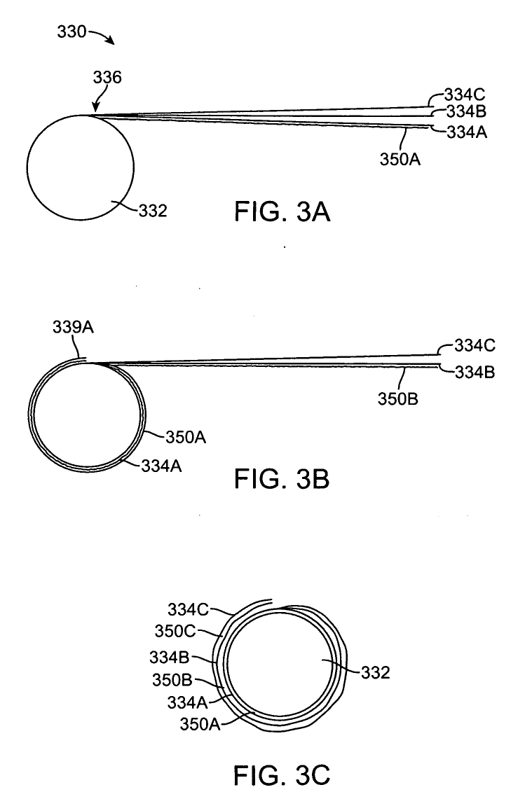

FIG. 4 illustrates a cross section of one embodiment of a stent framework 430, in accordance with the present invention. In this embodiment, stent framework 430 comprises a first stent foil 434A having a width that approximates the circumference of stent wire 432 and providing an exit port 454A adjacent first edge 436A when second edge 439A is secured to stent wire 432. Stent framework 430 further includes a second stent foil 434B having a width that is less than the circumference of stent wire 432. In this embodiment, the width of second stent foil 434B is approximately that of half the circumference of stent wire 432. The width of stent foil 434B provides an exit port 454B that is disposed opposite of exit port 454A. In this embodiment, the mean diffusion path for the therapeutic agent coated on stent foil 434A is greater than the mean diffusion path for the therapeutic agent coated on stent foil 435B. In this embodiment, when a stent formed of stent framework 430 is put in place at a treatment site, one exit port 454 is located to release the therapeutic agent into the vessel wall and the other exit port 454 is located to release the therapeutic agent into the vessel lumen. In an example, the therapeutic agent that is released at the vessel wall may be an anti-inflammatory agents and the therapeutic agent released into the vessel lumen may be an anticoagulant agent. Those with skill in the art will appreciate that the number, type and combination of drugs encompassed by the invention will depend on the particular application and is not limited to this specific combination.

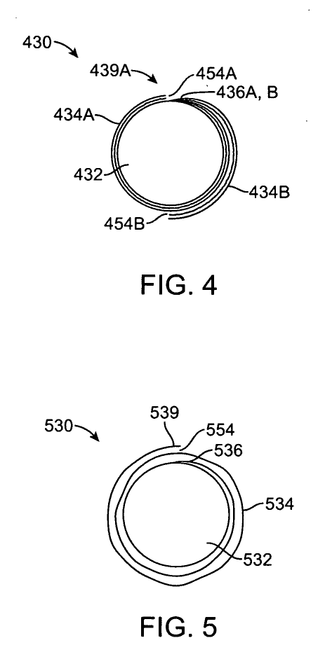

FIG. 5 illustrates a cross section of one embodiment of a stent framework 530, in accordance with the present invention. In this embodiment, stent framework 530 comprises stent foil 534 having a width that is approximately double the circumference of stent wire 532 and providing an exit port 554 adjacent first edge 536 when second edge 539 is secured to stent wire 532. In this embodiment, the mean diffusion path is effectively double the mean diffusion path for stent framework 230 described above and illustrated in FIG. 2C.

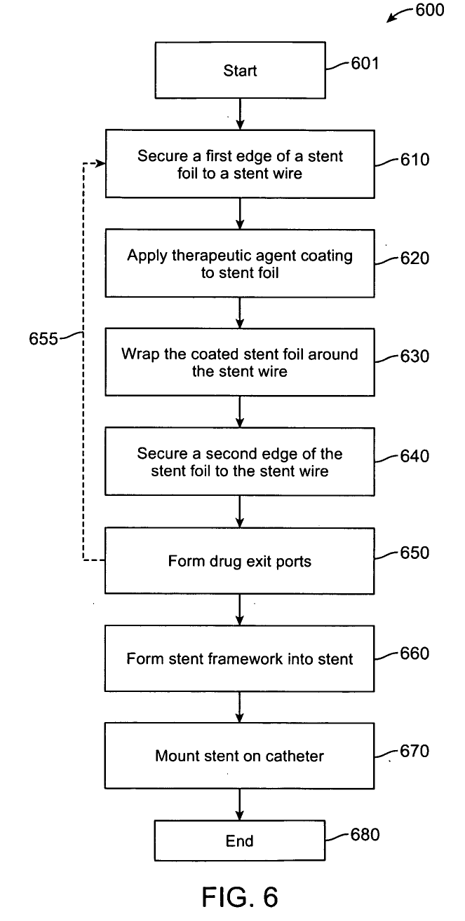

FIG. 6 is a flowchart of method 600 for manufacturing a stent having a foil wrapped stent framework, in accordance with the present invention. Method 600 begins at 601. Reference may be made to FIGS. 1 to 5 for embodiments manufactured using method 600. At block 610, a first edge 236 of at least one stent foil 234 is attached along the length of a stent wire 232. A therapeutic agent coating 250 is applied to an inner surface of the attached stent foil by dipping, brushing or spraying (Block 620). The therapeutic agent coating may be cured if necessary. Next, the coated stent foil is wrapped around the outer surface of stent wire 232 (Block 630). At Block 640 a second edge 239 of stent foil 234 is secured to stent wire 232. The second edge may be secured by a tack weld applied at intervals along the length of the stent wire. A plurality of exit ports are formed along the second edge between the dispersed tack welds (Block 650). In embodiments having more than one foil, method steps illustrated in Blocks 620 to 650 may be repeated as required (655). At block 660, the completed stent framework is formed into a stent. At block 670, the formed stent is mounted on a delivery catheter for use in treating a vascular condition. Method 600 ends at block 680.

In use, the drug delivery stent mounted at a distal end of a delivery catheter is inserted into a patient's vascular system and delivered to the treatment site. At the treatment site, the stent is positioned across the lesion to be treated and expanded. The catheter is then withdrawn from the body.

In the physiological environment, the therapeutic agents are released from the coating via the exit ports. The length of time for diffusion of the drugs is predetermined by the mean diffusion path of each respective therapeutic agent.

While the invention has been described with reference to particular embodiments for treating a vascular condition, it will be understood by one skilled in the art that variations and modifications may be made in form and detail without departing from the spirit and scope of the invention. In an example, the method of wrapping a wire with foil having a therapeutic agent coating may be applied to wires used for pacemaker leads as well as other leads and devices implanted into a patient's body.

1. A system for treating a vascular condition comprising:
a catheter;
a stent disposed on the catheter, the stent having a stent framework including a stent wire and at least one stent foil attached to and wrapped around the stent wire; and
a therapeutic agent coating disposed on an inner surface of the at least one stent foil.

2. The system of claim 1 wherein the at least one stent foil has a first edge attached to a first portion of an outer surface of the stent wire and a second edge attached to a second portion of the outer surface of the stent wire.

3. The system of claim 2 wherein the attachment of the second edge to a second portion of the outer surface provides at least one drug exit port between the outer surface of the stent wire and the second edge.

4. The system of claim 1 further comprising a first stent foil having a first therapeutic agent coating, a second stent foil having a second therapeutic agent coating and a third stent foil having a third therapeutic agent coating.

5. The system of claim 4 wherein the width of the first foil, second foil and the third foil is substantially equal to the circumference of the stent wire.

6. The system of claim 5 wherein the first therapeutic agent coating includes a first therapeutic agent, the second therapeutic agent coating includes a second therapeutic agent and the third therapeutic agent coating includes a third therapeutic agent.

7. The system of claim 6 wherein each of the therapeutic agents are different therapeutic agents.

8. The system of claim 1 further comprising a first stent foil having a first therapeutic agent coating and a second stent foil having a second therapeutic agent coating, the first stent foil having a first width and the second stent foil having a second width.

9. The system of claim 8 wherein the first width is greater than the second width and wherein a first edge of the first foil is secured between the surface of the stent wire and a second edge of the second stent foil.

10. The system of claim 1 wherein the at least one stent foil has a width that is greater than the circumference of the stent wire.

11. The system of claim 1 wherein the at least one therapeutic agent is selected from the group consisting of antirestenotic agents, anticoagulants, antiinflammatories, fibrinolytics, antiproliferatives, antibiotics, therapeutic proteins, recombinant DNA products, bioactive agents, diagnostic agents, radioactive isotopes, and radiopaque substances.

12. A stent for treating a vascular condition, the stent comprising:
a stent framework including a stent wire and at least one stent foil attached to and wrapped around the stent wire; and
a therapeutic agent coating disposed on an inner surface of the at least one stent foil.

13. The stent of claim 12 wherein the at least one stent foil has a first edge attached to a first portion of an outer surface of the stent wire and a second edge attached to a second portion of the outer surface of the stent wire.

14. The stent of claim 13 wherein the attachment of the second edge to a second portion of the outer surface provides at least one drug exit port between the outer surface of the stent wire and the second edge.

15. The stent of claim 12 further comprising a first stent foil having a first therapeutic agent coating, a second stent foil having a second therapeutic agent coating and a third stent foil having a third therapeutic agent coating.

16. The stent of claim 15 wherein the width of the first foil, second foil and the third foil is substantially equal to the circumference of the stent wire.

17. The stent of claim 16 wherein the first therapeutic agent coating includes a first therapeutic agent, the second therapeutic agent coating includes a second therapeutic agent and the third therapeutic agent coating includes a third therapeutic agent.

18. The stent of claim 12 further comprising a first stent foil having a first therapeutic agent coating and a second stent foil having a second therapeutic agent coating, the first stent foil having a first width and the second stent foil having a second width.

19. The stent of claim 18 wherein the first width is greater than the second width and wherein a first edge of the first foil is secured between the surface of the stent wire and a second edge of the second stent foil.

20. The stent of claim 12 wherein the at least one stent foil has a width that is greater than the circumference of the stent wire.

21. The stent of claim 12 wherein the at least one therapeutic agent is selected from the group consisting of antirestenotic agents, anticoagulants, antiinflammatories, fibrinolytics, antiproliferatives, antibiotics, therapeutic proteins, recombinant DNA products, bioactive agents, diagnostic agents, radioactive isotopes, and radiopaque substances.

22. A method of manufacturing a stent for treating a vascular condition, the method comprising:
securing a first edge of at least one stent foil to a stent wire;
applying a therapeutic agent coating to an inner surface of the at least one stent foil;
wrapping the therapeutic agent coated stent foil around an outer surface of the stent wire;
encasing the therapeutic agent coating between the inner surface of the stent foil and the outer surface of the stent wire;
securing a second edge of the wrapped stent foil to the stent wire;
forming at least one exit port along the secured second edge; and
forming the wrapped stent wire into a stent framework.
